(19) United States
(12) Patent Application Publication
(10) Pub. No.: US 2020/0339932 A1
(43) Pub. Date: Oct. 29, 2020

(54) Reactors for Modified Solid-State Fermentation

(71) Applicant: Locus IP Company, LLC, Solon, OH (US)

(72) Inventors: Sean FARMER, North Miami Beach, FL (US); Ken ALIBEK, Solon, OH (US); Tyler DIXON, Kent, OH (US); Nicholas CALLOW, Solon, OH (US); Timur ALIBEK, Solon, OH (US)

(21) Appl. No.: 16/962,306

(22) PCT Filed: Jan. 15, 2019
(86) PCT No.: PCT/US2019/013631

§ 371 Date: Jul. 15, 2020

Related U.S. Application Data
(60) Provisional application No. 62/617,425, filed on Jan. 15, 2018.

(51) Int. Cl. C12M 1/00; C12M 3/00; C12M 1/16; C12M 1/06; C12P 1/04; C12P 1/02

(52) U.S. Cl. CPC C12M 29/00; C12M 23/44; C12M 21/16; C12M 23/42; C12P 1/04; C12P 1/02; C12M 27/02

(57) ABSTRACT

The subject invention provides for continuous production of advantageous microbes and/or by-products using a system capable of submerged fermentation, solid-state fermentation, and combinations thereof. In particular, the systems utilize cassettes or other similar containers as vessels for holding a solid substrate inoculated with a microorganism. The cassettes are aligned inside a tank, and the tank can optionally be filled with a liquid nutrient medium. The cassettes with substrate and growing microorganisms are removed from the tank for harvesting the microorganism, and a new, sterilized cassette with substrate can be replaced into the tank.

### CROSS-REFERENCE TO RELATED APPLICATIONS

This application claims priority to U.S. Provisional Application No. 62/617,425, filed Jan. 15, 2018, which is incorporated herein by reference in its entirety.

### BACKGROUND OF THE INVENTION

Microorganisms, such as yeast, fungi and bacteria, are important for the production of a wide variety of useful bio-preparations in many settings, such as oil production; agriculture; remediation of soils, water and other natural resources; mining; animal feed; waste treatment and disposal; food and beverage preparation and processing; and human health.

One limiting factor, however, in commercialization of microbe-based products has been the cost per propagule density, where it is particularly expensive and unfeasible to apply microbial products to large scale operations with sufficient inoculum to see the benefits. This is partly due to the difficulties in cultivating efficacious microbial products on a large scale.

Two principle forms of microbe cultivation exist for growing bacteria, yeasts and fungi: submerged (liquid fermentation) and surface cultivation (solid-state fermentation (SSF)). Both cultivation methods require a nutrient medium for the growth of the microorganisms, but they are classified based on the type of substrate used during fermentation (either a liquid or a solid substrate). The nutrient medium for both types of fermentation typically includes a carbon source, a nitrogen source, salts and other appropriate additional nutrients and microelements.

In particular, SSF utilizes solid substrates, such as bran, bagasse, and paper pulp, for culturing microorganisms. One advantage to this method is that nutrient-rich waste materials can be easily recycled as substrates. Additionally, the substrates are utilized very slowly and steadily, so the same substrate can be used for long fermentation periods. Hence, this technique supports controlled release of nutrients. SSF is best suited for fermentation techniques involving fungi and microorganisms that require less moisture content; however, it cannot be used in fermentation processes involving organisms that require high water activity, such as certain bacteria.

Submerged fermentation, on the other hand, is typically better suited for those microbes that require high moisture. This method utilizes free flowing liquid substrates, such as molasses and nutrient broth, into which bioactive compounds are secreted by the growing microbes. While submerged cultivation can be achieved relatively quickly, it does possess certain drawbacks. For example, the substrates are utilized quite rapidly, thus requiring constant replenishment and/or supplementation with nutrients. Additionally, it requires more energy, more stabilization, more sterilization, more control of contaminants, and often a more complex nutrient medium than is required for SSF.

Microbes have the potential to play highly beneficial roles in, for example, the oil and agriculture industries; however, more efficient methods are needed for producing the large quantities of microbe-based products that are required for such applications.

### BRIEF SUMMARY OF THE INVENTION

This invention relates to the production of microbe-based products for commercial application. Methods are also provided for using these microbe-based products, for example, in enhanced oil recovery and/or agriculture. Advantageously, the subject invention can be used as a “green” process for producing microorganisms on a large scale and at low cost, without releasing harmful chemicals into the environment.

The subject invention provides systems and methods for the efficient production and use of beneficial microbes, as well as for the production and use of substances, such as metabolites, derived from these microbes and the substrate in, or on, which they are produced.

Advantageously, the subject system is a multi-use system, in that it can be used for solid state fermentation, submerged fermentation, and/or a combination thereof. Additionally, the system can be used for aerobic and/or anaerobic fermentation. Furthermore, the system can be scaled up or down in size. Most notably, the system can be scaled to an industrial scale, i.e., a scale that is suitable for use in commercial applications.

In one embodiment, a system for modified solid-state fermentation is provided, wherein the system comprises a tank. Preferably, the tank is a parallelepiped tank comprising four rectangular vertical walls, a horizontal bottom and a horizontal top. The front and back vertical walls of the tank are in parallel to one another, the left and right vertical walls are in parallel to one another, and the bottom and top of the tank are in parallel to one another.

The tank of the subject system can be made of, for example, polymers, glass, metal or metal alloys. In one embodiment, the tank is comprised of a combination of one or more metals and/or metal alloys. In one embodiment, the tank is comprised of stainless steel.

In certain embodiments, the bottom of the tank comprises a sealable outlet attached to a funnel that feeds into a drain and/or collection vessel.

In one embodiment, the bottom of the tank comprises an impeller for circulating liquid throughout the tank. In one embodiment, the bottom of the tank comprises a sparging system for injecting air and/or gases into the tank.

In some embodiments, the tank is fitted with an aeration system. In one embodiment, the aeration system can be attached to the sparging system to supply it with oxygen or other gases. If desired, an off-gas system can be used to remove, for example, carbon dioxide by-products.

In some embodiments, the system is fitted with ports and/or tubing through which liquids can be injected into the tank and/or removed from the tank. The ports and/or tubing can be used for injecting and/or removing, for example, liquid nutrient medium, water, and/or microbial inoculant.

When tubing is present, the tubing can be connected to one or more pumps, which facilitate the injection and/or removal of tank liquids.

In one embodiment, all or part of the top of the tank is removable. For example, the entire top of the tank can serve as a removable lid, or, simply a portion of the top can comprise a removable lid. The lid, when closed, effectively seals the tank.

In certain embodiments, the system further comprises one or more unsealed containers. In some embodiments, the containers are cassettes. “Cassettes,” as used herein, can also include baskets and/or trays, such as those utilized for instrument sterilization.

In one embodiment, the cassettes are fitted vertically into the tank through the top of the tank. The height and length of the cassettes can correspond to the interior dimensions of the tank, although in preferred embodiments, the width of the cassettes is about 0.5 inch to about 1.0 inch.

Preferably, the cassettes are comprised of material selected from silicon, polymer, metal, metal alloy, fibers, glass, or any combination thereof. In one embodiment, the cassettes are made of stainless steel.

In one embodiment, the cassettes comprise sides with a plurality of openings measuring about 5 mm or less in size. For example, the sides can comprise a mesh, netting or a screen, or a solid panel with holes therein. In one embodiment, the cassettes comprise a removable lid at the top.

In certain embodiments, the one or more cassettes are removable, meaning they are not permanently fixed inside the tank. In one embodiment, the cassettes slide vertically into and out of the tank through the top of the tank. Thus, in some embodiments, the inside surfaces of the left and right vertical walls of the tank comprise vertically-oriented grooves, guides, notches or tracks for sliding the cassettes into the tank and holding them in place.

In some embodiments, the vertically-oriented grooves, guides, notches or tracks are situated such that the cassettes are spaced apart from one another, in parallel, by a distance that is sufficient to allow fluids (e.g., gases, air and/or liquids) to circulate freely between them, or about 0.5 inch to about 1.0 inch apart. The number of cassettes that will fit inside the tank can vary depending upon the tank size, although preferably, the numbers ranges from 1 to 50, or from 2 to 25, or from 3 to 15.

In certain embodiments, the containers are formatted for holding a solid nutrient substrate in and/or on which microorganisms can grow.

The cassettes can be sterilized and/or filled with sold substrate prior to being placed into the tank. In some embodiments, the cassettes are placed into the system and then filled with solid substrate, sterilized, and inoculated in situ.

The system can be operated continuously, where one cassette having microbes growing therein is removed from the tank and replaced with a fresh, sterilized cassette having solid substrate placed therein.

Advantageously, the system does not require complicated temperature stabilization. For example, simply operating the system in a space or room having a stable, desirable temperature is sufficient.

In preferred embodiments, the subject invention provides a method of cultivating a microorganism and/or a microbial growth by-product using the subject system. The method can further be used to cultivate inocula for producing microbe-based products on an industrial scale.

The methods can comprise cultivation via solid state fermentation, submerged fermentation, or a combination thereof. Furthermore, the method can comprise aerobic and/or anaerobic fermentation.

In specific embodiments, the method comprises a) inserting one or more cassettes into a tank of the system of the subject invention; b) filling the one or more cassettes with a solid nutrient substrate (either before or after inserting the cassettes into the tank); c) seeding the system with an inoculant of the microorganism; d) sealing the tank; e) injecting air or a gas into the system; and f) cultivating the microorganism until a desired cell concentration and/or concentration of the microbial growth by-product is achieved.

The solid substrate preferably comprises solid foodstuff items, such as rice, beans, legumes, lentils, corn, grains, pasta, oats, oatmeal, wheat bran, wheat flour, corn flour, nixtamilized corn flour, corn meal, or partially hydrolyzed corn meal.

In some embodiments, the tank is filled with a liquid nutrient medium prior to sealing the tank.

Fermentation parameters can be optimized based on the desired product to be produced (e.g., the desired microbial growth by-product) and the microorganism being cultivated. For example, in the case of an anaerobic microorganism, the injected gas can be an inert gas, such as nitrogen.

In certain embodiments, when, for example, the microorganism prefers not to be cultivated in a liquid medium, the liquid medium can be drained after initial accumulation of microbial biomass. Circulation of warm air through the tank dries out the tank and the substrate, and then the method proceeds for the remainder of time via solid state fermentation.

The temperature is preferably kept between about 32-40° C. The culture can be grown for as long as necessary for the microorganism to reach the desired cell concentration and/or concentration of microbial growth by-products. For example, each individual cassette can be cultivated from 1 day to 2 weeks (14 days), or from 2 days to 10 days. In some embodiments, the methods can be used to produce a microbe culture having at least 1×10¹⁰ cells/gram.

Afterwards, the cassettes can be collected from the tank and the entire solid substrate with microorganism growing therein is harvested, blended into a slurry, and then, optionally, dried. Drying can be achieved by any known means, such as, e.g., using a rotary evaporator

In one embodiment, the dried product can be dissolved in water to form a liquid microbe-based product.

In one embodiment, the subject invention also provides methods of producing a growth by-product of a microorganism, wherein the method comprises cultivating the microorganism under conditions favorable for growth and production of the growth by-product, and optionally, extracting and/or purifying the growth by-product. In specific embodiments, the growth by-product is a biosurfactant, enzyme, biopolymer, bioemulsifier, acid, solvent, amino acid, nucleic acid, peptide, protein, lipid, carbohydrate, vitamin and/or mineral.

The growth by-product can be retained in the cells of the microorganisms and/or secreted into the solid substrate and/or liquid medium of the subject system.

In certain embodiments, the subject invention provides microbe-based products, as well as their uses in a variety of settings including, for example, oil and gas production; bioremediation and mining; waste disposal and treatment; animal health (e.g., livestock production and aquaculture); plant health and productivity (e.g., agriculture, horticulture, crops, pest control, forestry, turf management, and pastures); and human health (e.g., probiotics, pharmaceuticals and cosmetics).

Organisms that can be cultured using the materials and methods of the subject invention can include, for example, yeasts, fungi, bacteria, and archaea.

In some embodiments, the microorganisms are yeasts, such as, for example, Starmerella bombicola, Wickerhamomyces anomalus, Pseudozyma spp., Saccharomyces spp. or Pichia spp. yeasts. In some embodiments, the microorganisms are fungi, such as, for example, Trichoderma spp., as well as mushrooms such as Lentinula edodes (shiitake).

In some embodiments, the microorganisms are bacteria. The bacteria can be anaerobic, aerobic, microaerophilic, facultative anaerobes and/or obligate aerobes. In one embodiment, the bacteria are spore-forming bacteria. Non-limiting examples of bacteria include Bacillus spp. (e.g., Bacillus subtilis, Bacillus licheniformis, Bacillus amyloliquefaciens and Bacillus coagulans), Pseudomonas chlororaphis, Rhodococcus erythropolis and Azotobacter vinelandii.

The microbe-based products produced according to the methods of the subject invention can comprise the microorganisms themselves and/or their growth by-products, as well as residual growth medium and/or solid substrate. The microorganisms can be live, viable or in an inactive form. They can be in the form of vegetative cells, spores, conidia, hyphae, mycelia and/or a combination thereof. Furthermore, the microbes can be in the form of a biofilm.

Additional advantages to using the subject methods include reduced water and energy consumption; transportability and ease of use, even in remote areas; and simple collection of useful microbial products due to the fact that the microorganism is growing on a stationary, solid matrix.

### DETAILED DESCRIPTION

The subject invention provides systems and methods for the efficient production and use of beneficial microbes, as well as for the production and use of substances, such as metabolites, derived from these microbes and the substrate in, or on, which they are produced.

Advantageously, the subject system is a multi-use system, in that it can be used for solid state fermentation, submerged fermentation, and/or a combination thereof. Additionally, the system can be used for aerobic and/or anaerobic fermentation. Furthermore, the system can be scaled up or down in size. Most notably, the system can be scaled to an industrial scale, i.e., a scale that is suitable for use in commercial applications.

In specific embodiments, the system utilizes cassettes or other similar containers as vessels for holding a solid substrate inoculated with a microorganism. The cassettes are aligned inside a tank supplied with, for example, circulating liquid medium and/or oxygen or other gases.

### Selected Definitions

The systems and methods of the subject invention can be used to produce microbe-based compositions. As used herein, a “microbe-based composition” is a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. In preferred embodiments, the microbes are present, with medium in which they were grown, in the microbe-based composition. The cells may be present at, for example, a concentration of at least 1×10⁴, 1×10⁵, 1×10⁶, 1×10⁷, 1×10⁸, 1×10⁹, 1×10¹⁰, 1×10¹¹, 1×10¹² or 1×10¹³ or more cells per gram or milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise only a portion of the product of cultivation (e.g., only the growth by-products), and/or the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers, such as amino acids, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, a “biofilm” is a complex aggregate of microorganisms, wherein the cells adhere to each other and produce extracellular substances that encase the cells. Biofilms can also adhere to surfaces. The cells in biofilms are physiologically distinct from planktonic cells of the same organism, which are single cells that can float or swim in liquid medium.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of propagule) in association with a carrier.

In certain embodiments, purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material (e.g., glucose), an intermediate (e.g., acetyl-CoA) in, or an end product (e.g., n-butanol) of metabolism. Examples of a metabolite include, but are not limited to, a biosurfactant, enzyme, biopolymer, bioemulsifier, acid, solvent, amino acid, nucleic acid, peptide, protein, lipid, carbohydrate, vitamin and/or mineral.

As used herein, the term “plurality” refers to a quantity greater than one.

As used herein, the term “probiotic” refers to microorganisms, which, when administered in adequate amounts, confer a health benefit on the host. The probiotics may be available in foods and dietary supplements (e.g., yogurts, kefirs, capsules, tablets, and powders). In preferred embodiments, the microorganisms are live.

As used herein “reduction” means a negative alteration, and “increase” means a positive alteration, wherein the negative or positive alteration is at least 1%, 5%, 10%, 25%, 50%, 75%, or 100%.

As user herein, “reference” means a standard or control condition.

As used herein, “surfactant” means a surface-active compound that lower the surface tension (or interfacial tension) between two liquids or between a liquid and a solid. Surfactants act as, e.g., detergents, wetting agents, emulsifiers, foaming agents, and dispersants. A “biosurfactant” is a surface-active substance produced by a living cell.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Fermentation System Design and Operation

The subject invention provides systems and methods for the efficient production and use of beneficial microbes, as well as for the production and use of substances, such as metabolites, derived from these microbes and the substrate in, or on, which they are produced.

Advantageously, the system does not require complicated equipment or high energy consumption. The system can be portable and distributed to a location where the produced materials will be used. The microorganisms of interest can be cultivated at small or large scale onsite and utilized, even being still-mixed with their media. Similarly, the microbial metabolites can also be produced at large quantities at the site of need.

Furthermore, the system can be used to produce microbe-based products in remote locations. The microbe growth facilities may operate off the grid by utilizing, for example, solar, wind and/or hydroelectric power.

Advantageously, the subject system is a multi-use system, in that it can be used for solid state fermentation, submerged fermentation, and/or a combination thereof. Additionally, the system can be used for aerobic and/or anaerobic fermentation.

In one embodiment, a system for modified solid-state fermentation is provided, wherein the system comprises a tank. Preferably, the tank is a parallelepiped tank comprising four rectangular vertical walls, a horizontal bottom and a horizontal top. The front and back vertical walls of the tank are in parallel to one another, the left and right vertical walls are in parallel to one another, and the bottom and top of the tank are in parallel to one another.

The tank of the subject system can be made of, for example, polymers, glass, metal or metal alloys. In one embodiment, the tank is comprised of a combination of metals and/or metal alloys. In one embodiment, the tank is comprised of stainless steel.

Advantageously, the subject systems can be scaled depending on the intended use. For some smaller applications, the tank can be as small as 50 gallons, or even smaller. For applications where large volumes of culture are necessary, the system can be scaled to produce 20,000 gallons or more of product, for example, by using high volume tanks and/or by using a plurality of reactor systems that are, optionally, connected to one another by tubing, and, optionally, can be set up inside an enclosure or housing facility.

The tank of the subject system can range in size from a few liters to tens of thousands of liters. The tank can be, for example, from 5 liters to 5,000 liters or more, from 10 to 3,000 liters, from 20 to 2,000 liters, or from 30 to 1,000 liters.

In one embodiment, all or part of the top of the tank is removable. For example, the entire top of the tank can serve as a removable lid, or, simply a portion of the top can comprise a removable lid. The lid, when closed, effectively seals the tank.

In certain embodiments, the bottom of the tank comprises a sealable outlet attached to a funnel that feeds into a drain and/or collection vessel.

In one embodiment, the bottom of the tank comprises an impeller for circulating liquid throughout the tank. In one embodiment, the impeller is a standard four-blade Rushton impeller. In one embodiment, the impeller comprises an axial flow aeration turbine and/or a small marine propeller. In one embodiment, the impeller design comprises customized blade shapes to produce increased turbulence.

The system can further comprise an aeration system capable of providing filtered air or other gases to the culture. The aeration system can, optionally, have an air filter for preventing contamination of the culture. In certain embodiments, the unit can be equipped with a unique sparging system, through which the aeration system supplies air. Preferably, the sparging system comprises stainless steel injectors that produce microbubbles.

In an exemplary embodiment, the spargers can comprise from 4 to 10 aerators, comprising stainless steel microporous pipes (e.g., having a plurality of holes 1 micron or less in size), which are connected to an air supply. The unique microporous design allows for proper dispersal of gas bubbles throughout the culture, while preventing contaminating microbes from entering the culture through the air supply. Furthermore, in combination with the impeller, the sparging/aeration system provides a simple means for mixing and/or circulating fluids throughout the tank.

If desired, an off-gas system can be used to remove, for example, carbon dioxide or other gaseous by-products. The system can further include one or more vents (or pressure release valves (PRVs)).

In some embodiments, the system is fitted with ports and/or tubing, hoses or pipes through which liquids can be injected into the tank and/or removed from the tank. The ports and/or tubing can be used for injecting and/or removing, for example, liquid nutrient medium, water, and/or microbial inoculant.

When tubing is present, the tubing can be connected to one or more pumps, which facilitate the injection and/or removal of tank liquids. The flow rate can be, for example, from 10 to 20 to 200 gallons per minute.

In some embodiments, when liquid medium is left in the tank throughout some or all of the fermentation process, the pump(s) can operate continuously to circulate and/or replenish the liquid. The pump and/or pumps of the system can be sized to be suitable for establishing a recycle ratio (the volume pumped per hour/the total volume of reactor liquid) ranging from, for example, 30 to 0.10.

In certain embodiments, the system further comprises one or more unsealed containers. In some embodiments, the containers are cassettes. “Cassettes,” as used herein, can also include baskets and/or trays, such as those utilized for instrument sterilization.

In one embodiment, the cassettes are fitted vertically into the tank through the top. The height and length of the cassettes can correspond to the interior dimensions of the tank, although in preferred embodiments, the width of the cassettes is about 0.5 inch to about 1.0 inch.

Preferably, the cassettes are comprised of material selected from, for example, silicon, polymer, metal, metal alloy, fibers, glass, or any combination thereof. In one embodiment, the cassettes are made of stainless steel.

In certain embodiments, the cassettes are formatted for holding a solid nutrient substrate in and/or on which microorganisms can grow.

In one embodiment, the cassettes comprise sides with a plurality of openings measuring about 5 mm or less in size. These openings allow for liquid, nutrients and microorganisms to pass into the container and access the solid substrate, but do not allow the solid substrate to escape from the container. For example, the sides can comprise a mesh, netting or a screen, or a solid panel with micro-pores. In one embodiment, the cassettes comprise a removable lid at the top.

In certain embodiments, the one or more cassettes are removable, meaning they are not permanently fixed inside the tank. In one embodiment, the cassettes slide vertically into and out of the tank through the top of the tank. Thus, in some embodiments, the inside surfaces of the left and right vertical walls of the tank comprise vertically-oriented grooves, guides, notches or tracks for sliding the cassettes into the tank and holding them in place. In some embodiments, a latching mechanism can further hold the cassettes in place while they are present in the tank.

In some embodiments, the vertically-oriented grooves, guides, notches or tracks are situated such that the cassettes are spaced apart from one another, in parallel, by a distance that is sufficient to allow fluids (e.g., gases, air and/or liquids) to circulate freely between them, or about 0.5 inch to about 1.0 inch apart. The number of cassettes that will fit inside the tank can vary depending upon the tank size, although preferably, the numbers ranges from 1 to 50, or from 2 to 25, or from 3 to 15.

Advantageously, in one embodiment, the use of unsealed containers (e.g., cassettes or baskets), and the spacing between them obviates the need for sophisticated mixers or aeration systems, as the various surfaces of the containers are, naturally, readily accessible to being contacted with the fluids circulating inside the tank.

In preferred embodiments, a microbial inoculant is added to the system. In some embodiments, the inoculant comprises propagules of the desired microorganism, which can be prepared using any known fermentation method. In some embodiments, the propagules are spores.

In one embodiment, inoculation is performed by injecting the inoculum into a liquid medium in the tank through a port or tube, or by pouring, sprinkling or spraying the inoculum through the top of the tank. In one embodiment, the cassettes holding solid substrate are directly inoculated (e.g., via pipette), either prior to or after being placed into the tank.

Fermentation parameters can be optimized based on the desired product to be produced (e.g., the desired microbial biosurfactant) and the microorganism being cultivated.

Advantageously, the system does not require complicated temperature stabilization. For example, simply operating the system in a space or room having a stable, desirable temperature is sufficient. The system can, however, be adapted to ensure maintaining an appropriate fermentation temperature if it is desired. For example, the outside of the system can be reflective to avoid raising the system temperature during the day if being operated outdoors. The system can also be insulated so the fermentation process can remain at appropriate temperatures in low temperature environments. Any of the insulating materials known in the art can be applied including fiberglass, silica aerogel, ceramic fiber insulation, etc. The insulation can surround any and/or all of the tubes and/or tanks of the system.

A thermometer can be included and the thermometer can be manual or automatic. The thermometer can preferably be placed on any and/or all of the tanks of the reactor. An automatic thermometer can manage the heat and cooling sources appropriately to control the temperature throughout the fermentation process.

In one embodiment, the tanks may be able to monitor the growth of microorganisms inside the vessel (e.g., measurement of cell number and growth phases). Alternatively, a daily sample may be taken and subjected to enumeration by techniques known in the art, such as dilution plating technique. Dilution plating is a simple technique used to estimate the number of bacteria in a sample. The technique can also provide an index by which different environments or treatments can be compared.

In one embodiment, the tank comprises one or more sight glasses to view inside the tank without opening it.

The system can comprise an apparatus for reducing and/or preventing foam production, or the system can be supplied with de-foaming compounds. In some embodiments, the system is controlled by a touch screen programmable logic controller (PLC) with a completely automated interface, which can be used to monitor, for example, temperature, DO, and pH throughout fermentation.

Prior to microbe growth, the tanks may be disinfected or sterilized. In one embodiment, fermentation medium, air, and equipment are sterilized. The system may be connected to a sterilizing unit, e.g., an autoclave. The system may also have a sterilizing unit that sterilizes in situ before starting the inoculation.

In some embodiments, the entire system is sterilized prior to inoculation with a microorganism. This can be achieved by adding a liquid medium to the tank and bringing the liquid medium to a boil. Once the temperature inside the tank returns to a temperature suitable for the microorganism, the system can be inoculated.

In some embodiments, the cassettes are sterilized outside of the tank prior to being placed in the tank.

To harvest the microorganisms and/or their growth by-products after fermentation, any number of the cassettes are removed from the system through the top of the tank. For example, the entire system can be halted and all of the cassettes removed, or, the system can continue operating, and one cassette is removed at a time for harvesting.

In some embodiments, microorganisms and/or their growth by-products can further be harvested from the liquid nutrient medium.

In one embodiment, the system is a cascading system comprising a plurality of tanks, connected to one another via tubing. In the cascading system, the one or more pumps can have an input connected to the first tank via a first tube, and an output connected to the second tank via a second tube, and so on for as many tanks as are present in the system. The system can include one or more block valves (any generic valve used to stop flow) on the first tank and second tank inlets and outlets, and any and/or all of the tubes can have a check-valve for preventing backflow.

Once the first tank is completely full with liquid medium, the liquid therein can drain or be pumped into the second tank via the tubing. When the second tank is completely full with the nutrient medium and microbial inocula, the liquid is then pumped or drained into the third tank via the connective tubing, and so on.

### Submerged Surface Fermentation Design

In one embodiment, the system operates using principles of surface tension to create a hybrid “submerged surface fermentation” environment.

In addition to cassettes or baskets, the system can comprise one or more two-dimensional frames fitted vertically into the tank through the top. The dimensions of the frames can correspond to the interior dimensions of the tank. Preferably, the frames are comprised of a sturdy solid substance, such as, for example, metal, a metal alloy, a polymer, or glass.

Preferably, within each frame, a screen or netting made of, for example, stainless steel or another metal or metal alloy, thread, fiber, a polymer, or glass, is attached. The mesh of the screen or net is preferably 5 millimeters or less.

In one embodiment, the cassettes, baskets or frames are situated in parallel to one another. The cassettes, baskets or frames can be separated by a distance that is sufficient to allow fluids (e.g., air, gases and/or liquid) to circulate freely through and between them, for example, 0.5 in to 1 in. apart. The number of cassettes, baskets or frames inside the tank can vary depending upon the size of the tank. In certain embodiments, the number of frames ranges from 1 to 50, or from 2 to 25, or from 3 to 15.

When the one or more cassettes, baskets or frames are present inside the tank, the tank is filled with a liquid nutrient medium and then inoculated with cells and/or propagules (e.g., spores) of a microorganism. The liquid is then removed from the tank, leaving behind droplets of nutrient medium and microbial inoculant in the holes, spaces or mesh of the cassettes, baskets or frames due to the surface tension between the water and the mesh surfaces.

The cassettes, baskets or frames, inoculated with cells and/or propagules and nutrient medium, are then cultivated for 1 to 10 days at a stable temperature (typically room temperature). Each droplet can contain from 1 to 100 cells and/or spores, with each droplet serving as a miniature “reactor” for producing millions of microorganisms

In one embodiment, once the microorganisms have propagated to a desired concentration, the droplets can be collected at the bottom of the tank, harvested, and mixed to form a slurry. In one embodiment, harvesting can be achieved using a funnel attached to the bottom of the tank, which flows into a collection vessel. Optionally, the funnel can be attached to a vacuum for sucking the droplets into the collection vessel.

In one embodiment, collection of the droplets occurs by applying a rapid increase of pressure into the tank using a pump or air amplifier, which causes the droplets to fall to the bottom of the tank. In another embodiment, collection of the droplets occurs by physically, or manually, hitting, jolting, or slamming the cassettes, baskets or frames of the system with enough force to cause the droplets to fall to the bottom of the tank.

### Growth of Microbes According to the Subject Invention

The subject invention provides methods for cultivation of microorganisms and production of microbial metabolites and/or other by-products of microbial growth using solid state fermentation, submerged fermentation, or a combination thereof. As used herein “fermentation” refers to growth of cells under controlled conditions. The growth could be aerobic or anaerobic.

In one embodiment, the subject invention provides materials and methods for the production of biomass (e.g., viable cellular material), extracellular metabolites (e.g. small molecules, polymers and excreted proteins), residual nutrients and/or intracellular components (e.g. enzymes and other proteins).

In preferred embodiments, the subject invention provides a method of cultivating a microorganism and/or a microbial growth by-product using the systems of the subject invention. The method can further be used to cultivate inocula for producing microbe-based products on an industrial scale.

In specific embodiments, the method comprises a) inserting one or more cassettes into a tank of the system of the subject invention; b) filling the one or more cassettes with a solid nutrient substrate (this can be done either before or after inserting the cassettes into the tank); c) seeding the system with an inoculant of the microorganism; d) sealing the tank; e) injecting air or a gas into the system; and f) cultivating the microorganism until a desired cell concentration and/or concentration of the microbial growth by-product is achieved.

In some embodiments, the tank is filled with a liquid nutrient medium prior to sealing the tank. In some embodiments, the tank is filled with liquid nutrient medium prior to seeding the system with the inoculant. The liquid nutrient medium can be added in an amount that covers the entirety of the cassettes. When the liquid nutrient medium is left inside the tank throughout fermentation, a combination of solid-state and submerged fermentation takes place.

In certain embodiments, when, for example, the microorganism prefers not to be cultivated in a liquid medium, the liquid medium can be drained after initial accumulation of microbial biomass. Circulation of warm air through the tank dries out the tank and the substrate, and then the method proceeds for the remainder of time via solid state fermentation.

Alternatively, in one embodiment, no liquid medium is added at all, and the cassettes with solid substrate are inoculated manually in a dry tank. The process can then proceed as solid state fermentation.

The microorganisms can grow throughout the solid nutrient substrate, as well as in the liquid nutrient medium, if present. Furthermore, the microorganisms can produce microbial growth by-products, which can be extracted from the cells, the solid substrate and/or the liquid nutrient medium.

In one embodiment, the liquid nutrient medium comprises a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

In one embodiment, the liquid nutrient medium comprises a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, coconut oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the liquid nutrient medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included in the liquid medium. Usable inorganic salts can be potassium dihydrogen phosphate, dipotassium hydrogen phosphate, disodium hydrogen phosphate, magnesium sulfate, magnesium chloride, iron sulfate (e.g., ferrous sulfate heptahydrate), iron chloride, manganese sulfate, manganese sulfate monohydrate, manganese chloride, zinc sulfate, lead chloride, copper sulfate, calcium chloride, calcium carbonate, and/or sodium carbonate. These inorganic salts may be used independently or in a combination of two or more.

In some embodiments, the solid nutrient substrate comprises a plurality of individual solid items, e.g., pieces, morsels, grains or particles, that are, optionally, prepared by mixing with a liquid nutrient medium, salts and/or water. In preferred embodiments, the solid items are foodstuff. The foodstuff can include one or more of, for example, rice, beans, lentils, legumes, oats and oatmeal, corn and other grains, pasta, wheat bran, flours or meals (e.g., corn flour, nixtamilized corn flour, partially hydrolyzed corn meal), and/or other similar foodstuff to provide surface area for the microbial culture to grow and/or feed on.

In preferred embodiments, the solid nutrient substrate serves as a three-dimensional scaffold that provides ample surface area on which microbes can grow. In some embodiments, the methods allow for microbes to grow in the form of a biofilm. In some embodiments, the foodstuff in the matrix can also serve as a source of nutrients for the microbes.

In some embodiments, the method for cultivation may optionally comprise adding additional acids and/or antimicrobials into the substrate before and/or during the cultivation process.

In one embodiment, the equipment and materials for cultivation are sterilized prior to fermentation. The method can comprise, after filling the tank with liquid nutrient medium and prior to inoculating the system, boiling the liquid medium to sterilize the system, including the cassettes and solid substrate. After boiling, the inside of the tank is allowed to cool to a temperature suitable for the microorganism of interest (e.g., about 30 to 50° C.).

In some embodiments, the cassettes and solid substrate are sterilized outside of the tank prior to being placed in the tank. This can be achieved using, for example, an autoclave.

The microbial inoculant according to the subject methods preferably comprises propagules of the desired microorganism, which can be prepared using any known fermentation method. In some embodiments, the propagules are spores. The inoculant can be pre-mixed with water and/or a liquid nutrient medium, if desired.

In one embodiment, seeding the system with the microbial inoculant can be performed by pumping the inoculum into the tank through the tubing, or by pouring, sprinkling or spraying the inoculum into the tank through the top of the tank or through a port.

In one embodiment, seeding the system with the microbial inoculant can comprise inoculating the solid nutrient substrate directly by hand (e.g., using a pipette), either before or after filling the cassette.

Activation, or germination, of the microbes can be enhanced, at the time of inoculation, during cultivation or at the time of application, by adding L-alanine in low (micromolar) concentrations, manganese or any other known growth enhancer or stimulant.

Cultivation/fermentation parameters can be optimized based on the desired product to be produced (e.g., the desired microbial growth by-product) and the microorganism being cultivated.

The pH of cultivation should be suitable for the microorganism of interest, though advantageously, stabilization of pH using buffers or pH regulators is not necessary when using the subject cultivation methods.

In one embodiment, the method for cultivation of microorganisms is carried out at about 5° to about 100° C., preferably, 15 to 60° C., more preferably, 32 to 40° C. In a further embodiment, the cultivation may be carried out continuously at a constant temperature. In another embodiment, the cultivation may be subject to changing temperatures. Temperature can be kept within the preferred range by operating the system in an environment with a controlled, stable temperature.

The culture within each cassette can be grown for as long as necessary for the microorganism to reach the desired cell concentration and/or concentration of microbial growth by-products, preferably from 1 day to 2 weeks, more preferably, from 2 days to 10 days. In some embodiments, the methods can be used to produce a microbe culture having at least 1×10¹⁰ cells/gram.

After cultivation, the microbes and/or growth by-products can be collected from the system. In one embodiment, the cassettes are collected from the tank and the entire solid substrate infiltrated with microbial culture is emptied therefrom and blended into a slurry.

In one embodiment, the slurry can be dried to form granules or powder. Drying can be achieved by any known means, including, for example, using a rotary evaporator. If the rotary evaporator is used, the step of drying the slurry can comprise mixing the slurry with food grade starch or cellulose, adding a non-toxic solvent (e.g., ethyl acetate), placing the solvent and slurry mixture into the rotary evaporator, and operating the rotary evaporator to evaporate the solvent and form a dry powder.

Other known methods of drying, such as spray drying, freeze drying and/or lyophilization can be utilized as well. In one embodiment, the dried product can be dissolved in water to form a liquid microbe-based product.

Optionally, nutrients including, e.g., potassium (e.g., in the form of salts, 0.1% or lower), carbon (e.g., in the form of molasses and/or glucose 1-5 g/L), nitrates, extracts, amino acids, and others can be added to the water to enhance microbial viability. If desired, the microbe-based product can be diluted to a desired concentration, for example, 1×10⁶ to 1×10⁷ propagules/ml.

In one embodiment, the microorganisms and/or their growth by-products are harvested from the liquid nutrient medium using known methods, for example, centrifugation.

In one embodiment, the subject invention also provides methods of producing a growth by-product of a microorganism, wherein the method comprises cultivating the microorganism under conditions favorable for growth and production of the growth by-product, and optionally, extracting and/or purifying the growth by-product. In specific embodiments, the growth by-product is a biosurfactant, enzyme, biopolymer, bioemulsifier, acid, solvent, amino acid, nucleic acid, peptide, protein, lipid, vitamin, mineral and/or carbohydrate. The culture can comprise, for example, at least 1%, 5%, 10%, 20%, 30%, 40%, 50%, 60%, 70%, 80%, or 90% growth by-product.

The microbial growth by-product may be retained in the microorganisms or secreted into the solid nutrient substrate and/or liquid nutrient medium. In one embodiment, compounds for stabilizing the activity of microbial growth by-product can be added to the product.

The methods for cultivation of microorganisms and production of microbial by-products can be performed in a batch process or a continuous/quasi-continuous process.

In one embodiment, all of the culture-filled cassettes are removed upon completion of the cultivation (e.g., upon, for example, achieving a desired cell density, or density of a specified metabolite). In this batch procedure, an entirely new batch is initiated after sterilization of the system.

In another embodiment, only a portion of the cassettes is removed at any one time. In this embodiment, cassettes with growing culture remain in the tank, as well as liquid nutrient medium (if present initially) comprising viable cells. Fresh, sterilized cassettes having solid substrate therein can be placed into the vacated slot(s). If liquid medium is present in the tank, microbes present in the liquid medium can naturally inoculate the solid substrate in the newly placed container. Alternatively, the sterilized cassette can be inoculated manually either immediately before or immediately after being placed into the tank. In this manner, a continuous or quasi-continuous system is created.

### Microbial Strains Grown in Accordance with the Subject Invention

The microorganisms produced according to the subject invention can be, for example, bacteria, yeasts and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a yeast or fungus. Yeast and fungus species suitable for use according to the current invention, include Acaulospora, Aspergillus, Aureobasidium (e.g., A. pullulans), Blakeslea, Candida (e.g., C. albicans, C. apicola, C. batistae, C. bombicola, C. floricola, C. kuoi, C. riodocensis, C. stellate), Debaryomyces (e.g., D. hansenii), Entomophthora, Glomus (e.g., G. mosseae), Hanseniaspora (e.g., H. uvarum), Hansenula, Issatchenkia, Kluyveromyces, Lentinula edodes, Mortierella, Mucor (e.g., M piriformis), Penicillium, Phythium, Phycomyces, Pichia (e.g., P. anomala, P. guielliermondii, P. occidentalis, P. kudriavzevii), Pseudozyma (e.g., P. aphidis), Rhizopus, Rhodotorula (e.g., R. bogoriensis); Saccharomyces (S. cerevisiae, S. boulardii sequela, S. torula), Starmerella (e.g., S. bombicola), Torulopsis, Thraustochytrium, Trichoderma (e.g., T. reesei, T. harzianum, T. virens), Ustilago (e.g., U maydis), Wickerhamiella (e.g., W. domericqiae), Wickerhamomyces (e.g., W. anomalus), Williopsis, Zygosaccharomyces.

In one embodiment, the microorganism is a yeast characterized by its secretion of toxic proteins or glycoproteins (i.e., “killer toxins”), to which the strain itself is immune. These yeasts can include, but are not limited to species of, for example, Candida (e.g., C. nodaensis), Cryptococcus, Debaryomyces (e.g., D. hansenii), Hanseniaspora, (e.g., H. uvarum), Hansenula, Kluyveromyces (e.g., K. phaffii), Pichia (e.g., P. anomala, P. guielliermondii, P. occidentalis, P. kudriavzevii), Saccharomyces (e.g., S. cerevisiae), Torulopsis, Ustilago (e.g., U. maydis), Wickerhamomyces (e.g., W. anomalus), Williopsis (e.g., W mrakii), Zygosaccharomyces (e.g., Z. bailii), and others.

In one embodiment, the microorganisms are bacteria, including Gram-positive and Gram-negative bacteria, as well as some archaea. The bacteria may be, spore-forming, or not. The bacteria may be motile or sessile. The bacteria may be anaerobic, aerobic, microaerophilic, facultative anaerobes and/or obligate aerobes. Bacteria species suitable for use according to the present invention include, for example, Acinetobacter (e.g., A. calcoaceticus, A. venetianus); Agrobacterium (e.g., A. radiobacter), Azotobacter (A. vinelandii, A. chroococcum), Azospirillum (e.g., A. brasiliensis), Bacillus (e.g., B. amyloliquefaciens, B. firmus, B. laterosporus, B. licheniformis, B. megaterium, B. mucilaginosus, B. subtilis, B. coagulans), Chlorobiaceae spp., Dyadobacter fermenters, Frankia spp., Frateuria (e.g., F. aurantia), Klebsiella spp., Microbacterium (e.g., M. laevanifbrmans), Panioea (e.g., P. agglomerans), Pseudomonas (e.g., P. aeruginosa, P. chlororaphis, P. chlororaphis subsp. aureofaciens (Kluyver), P. putida), Rhizobium spp., Rhodospirillum (e.g., R. rubrum), Sphingomonas (e.g., S. paucimobilis), and/or Xanthomonas spp.

The microbes and their growth products produced according to the subject invention can be used to produce a vast array of useful products, including, for example, biopesticides, biosurfactants, ethanol, nutritional compounds, therapeutic compounds (e.g. insulin, vaccines), and biopolymers.

In certain embodiments, the microorganism is a yeast or fungus, such as, for example, Wickerhamomyces anomalus, Pichia guilliermondii (Meyerozyma guilliermondii), Pichia kudriavzevii (Wickerhamomyces kudriavzevii), Pichia occidentalis, Starmerella bombicola Pseudozyrna aphidis, Saccharomyces cerevisiae and/or Saccharomyces boulardii.

In one embodiment, the microorganism can be a fungus, such as a Trichoderma spp. fungus (e.g., T. harzianum, F viride, T. hamatum, and/or T. reesei), or a mushroom-producing fungus (e.g., Lentinula edodes (shiitake)).

In one embodiment, the microorganism is a bacteria, such as Pseudomonas spp. (e.g., P. aeruginosa, P. chlororaphis), Rhodococcus erythropolis, Azotobacter spp., Bacillus spp. bacterium (e.g., B. subtilis, B. licheniformis, B. firmus, B. laterosporus, B. megaterium, B. amyloliquefaciens and/or B. coagulans).

Other microbial strains including, for example, strains capable of accumulating significant amounts of useful metabolites, such as, for example, biosurfactants, enzymes and biopolymers, can be used in accordance with the subject invention.

### Compositions Produced According to the Subject Invention

The subject invention provides compositions comprising one or more microorganisms and/or one or more growth by-products thereof.

In one embodiment, the composition may comprise, for example, the one or more microorganisms along with any products of fermentation. In some embodiments, the products include microbial growth by-products, including, for example, biosurfactants, enzymes and/or other metabolites.

In one embodiment, the composition comprises the solid substrate containing the microorganism and/or the microbial metabolites produced by the microorganism and/or any residual nutrients. In some embodiments, the microbes of the composition are vegetative cells, or in spore, hyphae, mycelia and/or conidia form.

In one embodiment, the growth by-product is a biosurfactant. In one embodiment, the growth by-product is another microbial metabolite, including, for example, enzymes, biopolymers, bioemulsifiers, acids, solvents, amino acids, nucleic acids, peptides, proteins, lipid, carbohydrate, vitamins and/or minerals.

In some embodiments, the growth by-product is a biosurfactant. Biosurfactants are a structurally diverse group of surface-active substances produced by microorganisms. Biosurfactants are biodegradable and can be efficiently produced, according to the subject invention, using selected organisms on renewable substrates. Most biosurfactant-producing organisms produce biosurfactants in response to the presence of a hydrocarbon source (e.g. oils, sugar, glycerol, etc.) in the growing media. Other media components such as concentration of iron can also affect biosurfactant production significantly.

Microbial biosurfactants are produced by a variety of microorganisms such as bacteria, fungi, and yeasts. Exemplary biosurfactant-producing microorganisms include Starmerella spp. (e.g., S. bombicola), Pseudomonas spp. (e.g., P. aeruginosa, P. putida, P. florescens, P. fragi, P. syringae); Flavobacterium spp.; Bacillus spp. (e.g., B. subtilis, B. amyloliquefaciens, B. pumillus, B. cereus, B. licheniformis); Wickerhamomyces spp. (e.g., W. anomalus), Candida spp. (e.g., C. albicans, C. rugosa, C. tropicalis, C. lipolytica, C. torulopsis); Saccharomyces (e.g., S. cerevisiae); Pseudozyma spp. (e.g., P. aphidis); Rhodococcus spp. (e.g., R. erythropolis); Arthrobacter spp.; Campylobacter spp.; Cornybacterium spp.; Pichia spp. (e.g., P. guilliermondii, P. occidentalis); as well as others.

Biosurfactants are amphiphiles. They consist of two parts: a polar (hydrophilic) moiety and non-polar (hydrophobic) group. Due to their amphiphilic structure, microbial biosurfactants reduce the surface and interfacial tensions between the molecules of liquids, solids, and gases. This dynamic can be used to facilitate plant health, increase yields, manage soil aeration, and responsibly utilize available irrigation water resources.

Additionally, biosurfactants increase the surface area of hydrophobic water-insoluble substances, increase the water bioavailability of such substances. Biosurfactants accumulate at interfaces, thus reducing interfacial tension and leading to the formation of aggregated micellar structures in solution. The ability of biosurfactants to form pores and destabilize biological membranes permits their use as, e.g., antibacterial and antifungal agents.

Furthermore, biosurfactants are biodegradable, have low toxicity, are effective in solubilizing and degrading insoluble compounds in soil and can be economically produced using low-cost renewable resources. They can inhibit microbial adhesion to a variety of surfaces, prevent the formation of biofilms, and can have powerful emulsifying and demulsifying properties. Furthermore, biosurfactants can also be used to obtain wettability and to achieve even distribution of fertilizers, nutrients, and water in the soil.

Combined with the characteristics of low toxicity and biodegradability, biosurfactants can be useful in a variety of settings including, for example, oil and gas production; bioremediation and mining; waste disposal and treatment; animal health (e.g., livestock production and aquaculture); plant health and productivity (e.g., agriculture, horticulture, crops, pest control, forestry, turf management, and pastures); and human health (e.g., probiotics, pharmaceuticals, preservatives and cosmetics).

Thus, there exists an enormous potential for the use of microbial biosurfactants in a broad range of industries. One limiting factor in commercialization of these microbe-based products, however, has been the cost per propagule density, where it is particularly expensive and often unfeasible to cultivate efficacious microbial products on a large scale. Thus, the subject invention provides solutions to this problem through improved, scalable microbial fermentation systems and methods.

According to the subject invention, biosurfactants can include, for example, low-molecular-weight glycolipids, cellobiose lipids, lipopeptides, flavolipids, phospholipids, and high-molecular-weight polymers such as lipoproteins, lipopolysaccharide-protein complexes, and/or polysaccharide-protein-fatty acid complexes.

The hydrocarbon chain of a fatty acid acts as the common lipophilic moiety of a biosurfactant molecule, whereas the hydrophilic part is formed by ester or alcohol groups of neutral lipids, by the carboxylate group of fatty acids or amino acids (or peptides), or, in the case of glycolipids, by the carbohydrate.

In one embodiment, the biosurfactants according to the subject compositions comprise glycolipids and/or glycolipid-like biosurfactants, such as, for example, rhamnolipids (RLP), sophorolipids (SLP), trehalose lipids or mannosylerythritol lipids (MEL). In one embodiment, the biosurfactants comprise lipopeptides and/or lipopeptide-like biosurfactants, such as, e.g., surfactin, iturin, fengycin, athrofactin, viscosin and/or lichenysin. In one embodiment, the biosurfactants comprise polymeric biosurfactants, such as, for example, emulsan, lipomanan, alasan, and/or liposan.

In some embodiments, the pesticidal composition can comprise about 10 ppm to about 10,000 ppm of biosurfactant, or about 100 ppm to about 5,000 ppm, or about 200 to about 1,000 ppm, or about 300 ppm to about 800 ppm, or about 500 ppm.

In one embodiment, the microbial growth by-product is an enzyme. Enzymes are typically divided into six classes: oxidoreductases, transferases, hydrolases, lyases, isomerases and ligases. Each class is further divided into subclasses and by action. Specific subclasses of enzymes according to the subject invention include, but are not limited to, proteases, amylases, glycosidases, cellulases, glucosidases, glucanases, galactosidases, mannosidases, sucrases, dextranases, hydrolases, methyltransferases, phosphorylases, dehydrogenases (e.g., glucose dehydrogenase, alcohol dehydrogenase), oxygenases (e.g., alkane oxygenases, methane monooxygenases, dioxygenases), hydroxylases, esterases, lipases, ligninases, mannanases, oxidases, laccases, tyrosinases, cytochrome P450 enzymes, peroxidases (e.g., chloroperoxidase and other haloperoxidasese), and lactases. In exemplary embodiments, the enzyme is, for example, a phytase, a chitinase, a glucosidase and/or a glucanase (e.g., exo-(β-1,3-glucanase).

In one embodiment, the growth by-product is a biopolymer, such as, for example, levan, xanthan gum, alginate, hyaluronic acid, PGAs, PHAs, cellulose, and lignin.

In one embodiment, the microbial growth by-product is a protein, a lipid, a carbon source, an amino acid, a mineral or a vitamin.

In certain embodiments, the pesticidal composition can comprise the fermentation by-products containing a live and/or an inactive culture and/or the microbial metabolites produced by the microorganism and/or any residual nutrients. The product of fermentation may be used directly, with or without extraction or purification. If desired, extraction and purification can be easily achieved using standard extraction and/or purification methods or techniques described in the literature.

In one embodiment, the composition may comprise the medium in which the microbes were grown. The composition may be, for example, at least, by weight, 1%, 5%, 10%, 25%, 50%, 75%, or 100% growth medium. The amount of biomass in the composition, by weight, may be, for example, anywhere from 0% to 100% inclusive of all percentages therebetween.

The microorganisms may be in an active or inactive form, or in the form of vegetative cells, reproductive spores, mycelia, hyphae, conidia or any other form of microbial propagule. The composition may also contain a combination of any of these microbial forms.

In one embodiment, when a combination of strains of microorganism are included in the composition, the different strains of microbe are grown separately and then mixed together to produce the composition.

In one embodiment, the composition does not comprise living microorganisms. In one embodiment, the composition does not comprise microorganisms, whether living or inactive.

In one embodiment, the composition comprises the one or more microbial growth by-products separated from the microorganism that produced them. The growth by-products can be in a purified or unpurified form.

In certain embodiments, the compositions according to the subject invention can have advantages over, for example, purified microbial metabolites alone, due to, for example, the use of the entire culture. When producing yeasts, for example, the composition can comprise high concentrations of mannoprotein as a part of yeast cell wall's outer surface (rnannoprotein is a highly effective bioemulsifier). Additionally, the compositions can comprise a variety of microbial metabolites (e.g., biosurfactants, enzymes, acids, solvents, and other) in the culture that may work in synergy with one another to achieve a desired effect.

In certain other embodiments, the compositions comprise one or more microbial growth by-products, wherein the growth by-product has been extracted from the culture and, optionally, purified. For example, in one embodiment, the solid substrate and microorganisms can be harvested from the removal containers, blended to form a thick slurry that can be mixed with water or another solvent (e.g., saline), and filtered or centrifuged to separate the liquid portion from the solid portion. The liquid portion, comprising microbial growth by-products, can then be used as-is or purified using known methods.

### Methods of Use

The compositions of the subject invention can be used for a variety of purposes. In one embodiment, the composition can be used in agriculture. For example, methods are provided wherein a composition produced according to the subject invention is applied to a plant and/or its environment to treat and/or prevent the spread of pests and/or diseases. The composition can also be useful for enhancing water dispersal and absorption in the soil, as well as enhance nutrient absorption from the soil through plant roots, facilitate plant health, increase yields, and manage soil aeration.

In one embodiment, the subject compositions can be highly advantageous in the context of the oil and gas industry. When applied to an oil well, wellbore, subterranean formation, or to equipment used for recovery oil and/or gas, the compositions produced according to the subject invention can be used in methods for enhancement of crude oil recovery; reduction of oil viscosity; removal and dispersal of paraffin from rods, tubing, liners, and pumps; prevention of equipment corrosion; recovery of oil from oil sands and stripper wells; enhancement of fracking operations as fracturing fluids; reduction of H₂S concentration in formations and crude oil; and cleaning of tanks, flowlines and pipelines.

In one embodiment, the compositions produced according to the subject invention can be used to improve one or more properties of oil. For example, methods are provided wherein the composition is applied to oil or to an oil-bearing formation in order to reduce the viscosity of the oil, convert the oil from sour to sweet oil, and/or to upgrade the oil from heavy crude into lighter fractions.

In one embodiment, the compositions produced according to the subject invention can be used to clean industrial equipment. For example, methods are provided wherein a composition is applied to oil production equipment such as an oil well rod, tubing and/or casing, to remove heavy hydrocarbons, paraffins, asphaltenes, scales and other contaminants from the equipment. The composition can also be applied to equipment used in other industries, for example, food processing and preparation, agriculture, paper milling, and others where fats, oils and greases build up and contaminate and/or foul the equipment.

In one embodiment, the compositions produced according to the subject invention can be used to enhance animal health. For example, methods are provided wherein the composition can be applied to animal feed or water, or mixed with the feed or water, and used to prevent the spread of disease in livestock and aquaculture operations, reduce the need for antibiotic use in large quantities, as well as to provide supplemental proteins and other nutrients.

In one embodiment, the compositions produced according to the subject invention can be used to prevent spoilage of food, prolong the consumable life of food, and/or to prevent food-borne illnesses. For example, methods are provided wherein the composition is applied to a food product, such as fresh produce, baked goods, meats, and post-harvest grains, to prevent undesirable microbial growth.

Other uses for the subject compositions include, but are not limited to, biofertilizers, biopesticides, bioleaching, bioremediation of soil and water, pharmaceutical adjuvants (for increasing bioavailability of orally ingested drugs), cosmetic products, control of unwanted microbial growth, and many others.

### Preparation of Microbe-Based Products

One microbe-based product of the subject invention is simply the slurry containing the microorganism and/or the microbial metabolites produced by the microorganism and/or any residual nutrients. Upon harvesting of the slurry, the product can be easily dried and then optionally, dissolved in water, e.g., in a storage tank. If a mixture of microbial species is desired in a final product, the microbes are preferably grown in separate reactors and then mixed together after being harvested and/or dried.

The product of fermentation may be used directly without extraction or purification. If desired, extraction and purification can be easily achieved using standard extraction methods or techniques known to those skilled in the art.

The microorganisms in the microbe-based product may be in an active or inactive form. In some embodiments, the microorganisms have sporulated or are in spore form. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

In one embodiment, the solution containing the dissolved culture is diluted to a concentration of, for example, 1×10⁶ to 1×10⁷ cells/mL using water (and further added nutrients, if desired) to form a liquid microbe-based product, which can be utilized in a wide variety of settings and applications.

The microbes and/or liquid product containing the dissolved culture can be removed from the storage tank and transferred to the site of application via, for example, tanker for immediate use.

In other embodiments, the composition (in the form of solid substrate or in dissolved liquid form) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In certain embodiments the containers are 2 gallons, 5 gallons, 25 gallons, or larger.

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, pesticides, and other ingredients specific for an intended use.

Advantageously, in accordance with the subject invention, the microbe-based product may comprise the substrate in which the microbes were grown. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., an oil well). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used, for example, within 300 miles, 200 miles, or even within 100 miles. Advantageously, this allows for the compositions to be tailored for use at a specified location. The formula and potency of microbe-based compositions can be customized for a specific application and in accordance with the local conditions at the time of application.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

1. A system for cultivating a microorganism and/or producing a microbial growth by-product, wherein the system comprises a tank, said tank comprising:
a front vertical wall and a back vertical wall in parallel to one another,
a left vertical wall and a right vertical wall in parallel to one another,
a horizontal bottom and a horizontal top in parallel to one another,
wherein said tank comprises one or more removable containers fitted vertically therein, and wherein said removable containers are cassettes or instrument sterilization baskets.

2. canceled

3. The system of claim 1, wherein the tank is fitted with tubing through which liquids are injected into the tank and/or removed from the tank.

4. The system of claim 3, wherein the tubing is connected to a pump to power the injection and/or removal of liquids.

5. The system of claim 1, wherein the tank is comprised of one or more metals, metal alloys, polymers, or glass.

6. canceled

7. The system of claim 1, wherein the inside surfaces of the left and right vertical walls comprise vertically-oriented grooves, guides, notches or tracks for holding the removable containers in place, and
wherein the vertically-oriented grooves, guides, notches or tracks are situated such that the removable containers are spaced about 0.5 inch to about 1.0 inch apart from one another.

8. The system of claim 1, wherein the removable containers comprise sides with a plurality of openings measuring 5 mm or less in size.

9-10. canceled

11. The system of claim 1, wherein the removable containers are about 0.5 to about 1 inch thick.

12-13. canceled

14. The system of claim 1, wherein the tank bottom comprises an impeller.

15. The system of claim 1, comprising a sparging system and/or an aeration system.

16-17. canceled

18. A method for cultivating a microorganism and/or a microbial growth by-product using a system of claim 1, the method comprising:
inserting one or more cassettes into the tank;
filling the one or more cassettes with a solid nutrient substrate;
seeding the system with an inoculant of the microorganism;
sealing the tank;
injecting air or another gas into the system; and
cultivating the microorganism until a desired cell concentration and/or concentration of the microbial growth by-product is achieved.

19-23. canceled

24. The method of claim 18, wherein the microbial inoculant comprises cells and/or propagules of a bacteria, yeast or fungus.

25. The method of claim 24, wherein the bacteria is Bacillus subtilis, Bacillus licheniformis, Bacillus amyloliquefaciens, Bacillus coagulans, Bacillus firmus, Bacillus laterosporus, Bacillus megaterium, Pseudomonas chlororaphis, Rhodococcus erythropolis and/or Azotobacter vinelandii.

26. The method of claim 24, wherein the yeast is Wickerhamomyces anomalus, Pichia guilliermondii (Meyerozyma guilliermondii), Pichia kudriavzevii, Pichia occidentalis, Starmerella bombicola, Pseudozyma aphidis, Saccharomyces cerevisiae and/or Saccharomyces boulardii.

27. The method of claim 24, wherein the fungus is Trichoderma harzianum, Trichoderma viride, Trichoderma hamatum, Trichoderma reese, and/or Lentinula edodes.

28-30. canceled

31. The method of claim 18, wherein cultivation of each individual cassette occurs for 1 to 14 days.

32. The method of claim 18, further comprising collecting the microorganisms and/or microbial growth by-products from the system.

33. The method of claim 32, wherein collecting comprises removing one or more cassettes from the tank; emptying the solid substrate with microbial culture therefrom; and blending the solid substrate and microbial culture to form a slurry.

34. canceled

35. The method of claim 31, wherein all of the cassettes are removed at one time and an entirely new batch of cassettes are placed into the tank and filled with solid nutrient substrate.

36. The method of claim 31, wherein one cassette is removed from the tank at a time and a new, sterilized cassette filled with solid nutrient substrate is placed into the tank where the removed cassette was located.

37. The method of claim 18, wherein the growth by-products are selected from biosurfactants, enzymes, biopolymers, bioemulsifiers, acids, solvents, amino acids, nucleic acids, peptides, proteins, lipid, carbohydrate, vitamins and minerals.

38-39. canceled
